(19) United States
(12) Patent Application Publication
(10) Pub. No.: US 2026/0224769 A1
(43) Pub. Date: Aug. 6, 2026

(54) DEVICE AND METHOD FOR CAPTURING AND/OR NEUTRALIZING PATHOGENS IN AIR

(71) Applicant: Ohio University, Athens, OH (US)

(72) Inventors: Khairul Alam, Athens, OH (US); Muhammad Alli, Pickerington, OH (US); Sean R. Jenson, Athens, OH (US)

(21) Appl. No.: 19/146,422

(22) PCT Filed: Jan. 9, 2024
(86) PCT No.: PCT/US2024/010832

§ 371 Date: Jul. 9, 2025

Related U.S. Application Data
(60) Provisional application No. 63/479,389, filed on Jan. 11, 2023.

(51) Int. Cl. A61L 9/12

(52) U.S. Cl. CPC A61L 9/12; A61L 2209/16

(57) ABSTRACT

A device, system, and method for neutralizing pathogens in air flow. The device includes disinfectant-generating reactants (a disinfectant catalyst) within an outer layer. In use, liquid (such as water) is delivered to the disinfectant catalyst/outer layer modules. In an embodiment, the supplied liquid and disinfectant-generating reactants (of the disinfectant catalyst) combine, and generation of a disinfectant commences. The disinfectant in vapor phase is generated and diffuses through the outer layer of the module and is delivered to the air flow within an apparatus or system (e.g., HVAC system, electrostatic precipitator, etc.).

### CROSS-REFERENCE TO RELATED APPLICATIONS

This application claims priority to, and the benefit of the filing date of, U.S. application Ser. No. 63/479,389, filed Jan. 11, 2023, the disclosure of which is incorporated by reference herein in its entirety.

### TECHNICAL FIELD

The present invention relates generally to devices and methods of treating air and, more particularly, to the use of a disinfectant for treating air via capture and removal of contaminants, such as pathogens, therefrom.

### BACKGROUND

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

As is known, many air sources may include contaminants that can be harmful or potentially harmful to individuals who are exposed to the contaminants. Such contaminants may include biological pathogens (such as viruses and bacteria), chemical contaminants (such as may be found in gaseous pollutants), and particulate emissions. Because such contaminants can present health hazards when encountered by humans and animals (and/or because they can be disruptive and harmful to inanimate objects, such as equipment- or damaging to the environment), it is important to remove these contaminants.

To that end, traditional precipitator devices, such as electrostatic precipitators, and scrubbers are widely used for treating air sources (such as exhaust) containing gaseous pollutants and/or particulate emissions. For example, industrial processes capable of discharging exhaust containing gaseous pollutants at an elevated temperature may also be fitted with a scrubber and/or a wet electrostatic precipitator (“wet ESP”) to both remove gaseous pollutants, such as particulate emissions, and recover thermal energy. Wet electrostatic precipitators typically include a liquid, such as water, to capture both particulate and gaseous emissions as well as thermal energy, which may be directed through a heat exchanger for improved efficiency.

While electrostatic precipitators, scrubbers, and heat exchangers are generally known for use in removing gaseous pollutants and particulate emissions, they are generally not used for (or capable of efficient use) in removing biological contaminants, such as viruses and bacteria. While some wet precipitators may integrate a disinfectant solution as the liquid (or as part of the liquid) that flows through, along, and/or in association with the liquid supply conduit, sieves, etc. of the precipitator, the use of these disinfectant solutions has not proven useful or efficient in removing pathogens- and they can be cumbersome in use. Alternative equipment (other than precipitators) has been developed for attempted use in removal of such biological contaminants; however, such devices also do not operate at a high level of efficiency and/or are complex, cumbersome, come at an increased cost, etc.

Thus, there is a need for a device and method of treating air that improves treatment effectiveness, reduces complexity, reduces costs, and addresses present challenges and characteristics in the removal of biological pathogens.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

One aspect of the present invention is directed to a module including a disinfectant-generating reactant (which may be referred to herein as a disinfectant catalyst). The disinfectant catalyst may be at least partially disposed within an outer layer (such as a membrane sleeve). In an aspect of the present invention, a liquid (e.g., water) may be introduced to the module, and the combination of the disinfectant catalyst with the liquid generates a disinfectant, which then may neutralize pathogens. Once generated, the disinfectant may be present at least partially in a vapor phase in the air in the vicinity of the module. As such, the disinfectant may neutralize pathogens in the air in the vicinity of the module. Additionally, the disinfectant may also neutralize pathogens that impact the surface of the module (or any other surface onto which the generated disinfectant may settle).

Another aspect of the present invention includes a plurality of modules (each including a disinfectant catalyst) arranged within a housing. The housing may include conduits that communicate with the modules. The conduits may operate as a liquid delivery component to transport a liquid into contact with one or more of the modules, to generate a disinfectant therefrom.

The module(s) or plurality of modules arranged within a housing may be introduced into any type of system to treat air within the particular system. For example, such systems where the modules may be used include (but are not limited to) HVAC systems or any air filtration type system (such as electrostatic precipitators). By positioning the modules within the airflow path of such systems, one may cause capture and/or neutralization of pathogens in the airflow.

Another aspect of the present invention is directed to the geometries of the modules. In a general aspect, the modules (or the components thereof such as the disinfectant catalyst and any outer layer) may be of a shape that allows for increased surface area of each module, and allows for vibration of the module. Due to vibrations (which may be caused by the passage of airflow around each module), the area of the plume of disinfectant vapor phase that is shed from the module may be increased, thus leading to more efficient neutralization of pathogens. Increased surface area may also contribute to an increased amount of disinfectant that is shed into the airflow- and provides additional surface that can be impacted by pathogens in the airflow. Additionally, the geometries of the modules can also contribute to increased efficiency (e.g., irregular shapes can create more turbulence around the modules during vibration). This results in a greater dispersion of disinfectant in the vapor phase. The various geometries that may be used to increase efficiency may be geometries of the disinfectant catalyst, or of the outer layer, or both.

### BRIEF DESCRIPTION OF THE DRAWINGS

The accompanying drawings, which are incorporated in and constitute a part of this specification, illustrate embodiments of the invention and, together with a general description of the invention given above, and the detailed description given below serve to explain the invention.

FIGS. 1A and 1B depict a panel structure having a plurality of disinfectant catalyst modules.

FIG. 1C depicts a single disinfectant catalyst module (used in the panel embodiment of FIGS. 1A and 1B) with a cutaway view of an outer sleeve to show the inner disinfectant catalyst.

FIGS. 2A and 2B depict an alternate embodiment of a single disinfectant catalyst module with a cutaway view of an outer sleeve to show the inner disinfectant catalyst.

FIGS. 3A and 3B depict a panel structure having a plurality of disinfectant catalyst modules, and yet another alternate embodiment of a single module with a cutaway view of an outer sleeve to show the inner disinfectant catalyst.

FIGS. 4A and 4B depict a panel structure having a plurality of disinfectant catalyst modules, and yet another alternate embodiment of a single module with a cutaway view of an outer sleeve to show the inner disinfectant catalyst.

FIGS. 5A and 5B shows an outer layer of a module, and a cross section thereof, in non-filled form.

FIGS. 5C and 5D shows an outer layer of a module, and a cross section thereof, in filled form.

FIG. 6 is a perspective view of a first exemplary embodiment of a device for treating an air flow having a plurality of sieve assemblies.

FIG. 7 is an overhead plan view of the apparatus of FIG. 6 in a duct.

FIG. 8 is an overhead plan view of a second exemplary embodiment of a device for treating an airflow having an array of sieves.

FIG. 9A is a top sectional view of an airflow flowing along a module of a module assembly with the module in an initial transverse position.

FIG. 9B is a top sectional view of the airflow and the module similar to FIG. 9B, but showing the module in a terminal transverse position.

FIG. 10A is a diagrammatic depiction of a stationary string and a vibrating string showing the straight line motion of the vortices shedding off the stationary cylinder.

FIG. 10B is a diagrammatic depiction of a plurality of vibrating modules showing the cylinder vibrating in one direction with the vortex swayed in the opposite direction; in the next vibrating cycle, the motion directions of the cylinder and the vortex are changed

FIG. 10C is a diagrammatic depiction showing the capture of pathogens by impaction and by flowing close to the vibrating cylinders and taken by the swayed vortices and captured and neutralized.

### DETAILED DESCRIPTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

Various aspects of the present invention provide a device and method of treating air that improves treatment effectiveness, reduces complexity, reduces costs, and addresses present challenges and characteristics in the removal of biological pathogens.

To that end, and as described briefly above, one aspect of the present invention is directed to a module that can be used by itself, as part of an apparatus, and/or within existing systems to remove pathogens from air via capture and/or neutralization. The module includes a disinfectant-generating reactant (which may be referred to herein as a disinfectant catalyst). The disinfectant catalyst may be at least partially disposed within an outer layer (such as a membrane sleeve). In an aspect of the present invention, a liquid (e.g., water) may be introduced to the module, and the combination of the disinfectant catalyst with the liquid generates a disinfectant, which then may neutralize pathogens. Once generated, the disinfectant may be present at least partially in a vapor phase in the air in the vicinity of the module. As such, the disinfectant may neutralize pathogens in the air in the vicinity of the module. Additionally, the disinfectant may also neutralize pathogens that impact the surface of the module (or any other surface onto which the generated disinfectant may settle).

Another aspect of the present invention includes a plurality of modules (each including a disinfectant catalyst) arranged within a housing. The housing may include conduits that communicate with the modules. The conduits may operate as a liquid delivery component to transport a liquid into contact with one or more of the modules, to generate a disinfectant therefrom.

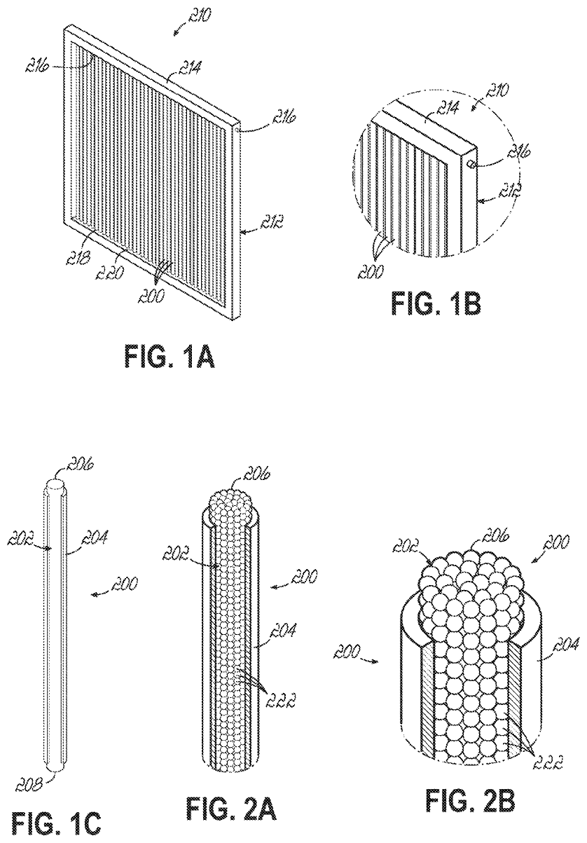

More specifically, and referring now to FIGS. 1A-1C, a module 200 (or modules), in accordance with the principles of the present invention, include a disinfectant-generating reactant 202 (which may be referred to herein as a disinfectant catalyst 202 or disinfectant catalysts) at least partially disposed within an outer layer 204 (such as a permeable membrane sleeve). In particular, the illustrated embodiment of FIG. 1C includes a solid disinfectant catalyst core encased in an outer layer (e.g., an elastic polypropylene mesh outer layer). As described above, the introduction of a liquid-such as water to the catalyst can generate a disinfectant, which may then be present on the surface of the module and/or in vapor phase in the area surrounding the module (as will be described in greater detail below).

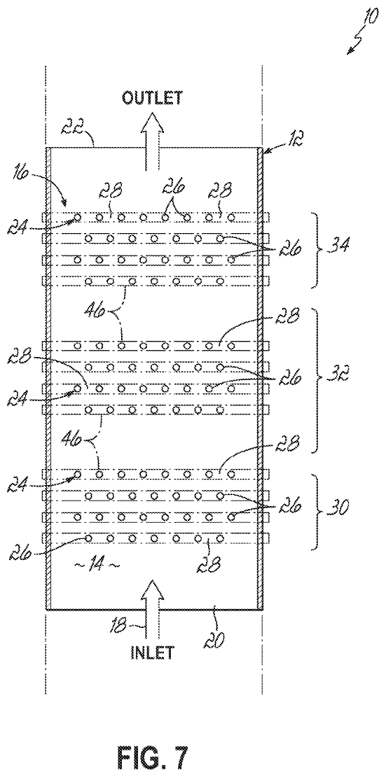

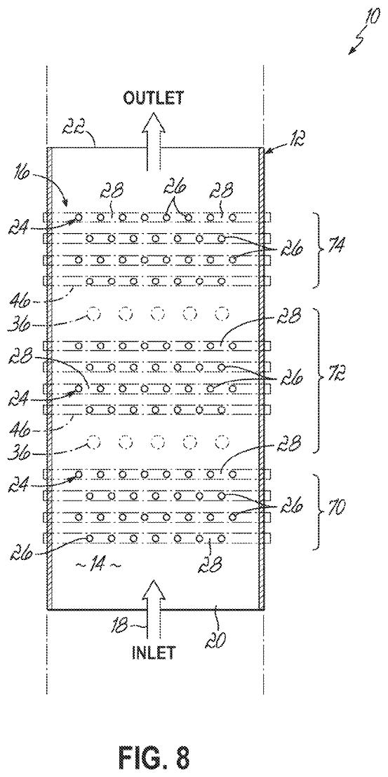

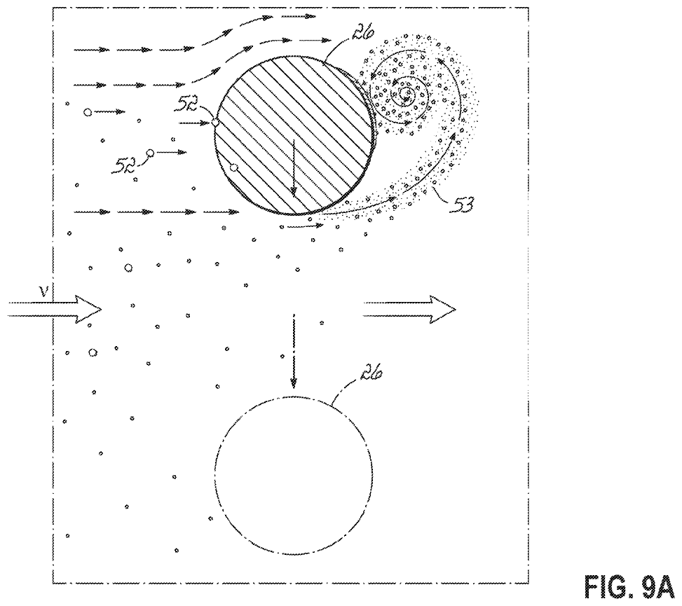

This module, or a plurality of such modules (including disinfectant catalyst 202 and outer layer 204) can be used in any number of systems where one may wish to reduce and eliminate pathogens, such as viruses or bacteria. One such system is an HVAC system. Another such system is a precipitator device (such as the precipitator device 10 shown in FIGS. 6 and 7).

While one, or at least one module, may be used—in another embodiment a plurality of disinfectant catalyst/outer layer modules 200 may be provided as part of a panel structure 210 that can be obtained as a separate component and inserted into an existing precipitator for use (or inserted into other apparatus for use in treating exhaust, air, etc.). Such a panel structure 210 is shown in FIGS. 1A and 1B. In various embodiments, such a panel structure 210 may be placed in an apparatus or system (such as a precipitator or HVAC system), and then removed once the disinfectant supply is exhausted. A new panel structure including fresh disinfectant supply may then replace the exhausted panel structure. (It will be recognized by those of ordinary skill in the art that, while a panel structure 210 having a single row of disinfectant catalyst/outer layer modules 200 is shown in FIGS. 1A and 1B, the present invention is not limited to this particular configuration, and other configurations are possible. In alternate embodiments, individual modules may be removed and replaced once the disinfectant catalyst is depleted.)

Referring still to FIGS. 1A and 1B, the panel structure 210 includes a housing comprising an outer frame 212, which includes a generally horizontally extending top support member 214. A liquid supply conduit 216 may be associated with frame 212 such as by being positioned adjacent to frame, or by being defined by frame by extending therethrough (e.g., by extending through top support member). The top support member 214 may help support the generally vertical orientation of the modules 200 and is configured to introduce liquid in the liquid supply conduit 216 to the module or modules 200. In certain embodiments, the support member 214 and the liquid supply conduit 216 may be in the collective form of a single elongate tube; however, it will be appreciated that many other designs for supporting the modules and providing for the supply of liquid to the modules may be used.

In a particular embodiment of the version shown in FIG. 1C, the disinfectant catalyst/outer layer module may include a solid disinfectant catalyst (in the shape of a rod, for example) at least partially encased in a mesh outer layer (such as a polypropylene mesh outer layer).

And, according to the illustrated embodiment of FIGS. 1A and 1B, an inlet 216 and an outlet 218 are positioned at opposing end portions of the disinfectant catalyst/outer layer modules 200, such as a top end and bottom end thereof. However, it will be appreciated that the inlet and outlet may alternatively include or additionally include further structures, which may define, respectively, the inlet and outlet.

Thus, in one exemplary embodiment, as liquid is introduced (via liquid supply conduit) to module proximal to the top end of the module, it flows down module (via gravity and/or capillary action). As it does so, it can pass through outer layer (for example, outer layer may be a permeable mesh). As it does so, it will contact the disinfectant catalyst, thereby generating disinfectant. The liquid will continue to flow down the module until it reaches outlet proximal to the bottom end of the module.

A liquid collector 220 may be positioned proximate to the outlets 218 for collecting the liquid being discharged from the outlets. In addition, the disinfectant catalyst/outer layer modules 200 may connect to the liquid collector 220 as part of the outer support of the panel structure. And the liquid collector 220 may be configured to guide the liquid to a liquid treatment system. As such, the disinfectant catalyst/outer layer modules 200 extend between portions of the panel structure. In certain embodiments, the disinfectant catalyst/outer layer modules are configured to vibrate by passing the flow of the air transversely therealong. Alternatively, the disinfectant catalyst/outer layer modules 200 may be operatively connected to a vibration mechanism configured to actively vibrate the disinfectant catalyst/outer layer modules 200 during use. The vibration will be described in greater detail below.

Alternatively to the particular arrangement shown in FIGS. 1A and 1B, the liquid collector 220 may be in the form of a tray 56 that includes a bottom 58 and surrounding sidewalls 60 configured to guide the liquid to a liquid treatment system 62 (such as that shown in FIG. 6—which shows a precipitator as a nonlimiting example of a device or system that the modules can be used with).

Regardless of the embodiment of liquid collector, the liquid treatment system 62 (as in FIG. 6) may include a pump 64, a filtration system 66, and a heat exchanger 68. The pump 64 is configured to direct the liquid from the liquid collector 56 to the filtration system 66, which is configured to remove particulate emissions from the liquid. The pump 64 then continues to direct the liquid through the heat exchanger 68 for recovering thermal energy from the liquid. While the liquid may be removed from the device 10 or system, the liquid may also be redirected back into the liquid supply conduit 46 for reuse through the disinfectant catalyst/outer layer modules 200. It will be appreciated that the pump 64, filtration system 66, and heat exchanger 68 may be selected and assembled in order to accommodate any performance requirements for treating the exhaust of any given industrial process. For this reason, the pump 64, filtration system 66, and heat exchanger 68 may be selected and assembled per known requirements readily appreciated by those having ordinary skill in the art. Again, the liquid treatment system described here is merely exemplary, and the use of the modules 200 in association with a precipitator is merely an example.

In use, liquid (such as water) is delivered to the liquid supply conduit 216 and directed to the disinfectant catalyst/outer layer modules 200. This can be accomplished by the liquid passing through inlets to contact and flow along the disinfectant catalyst/outer layer modules 200. The supplied liquid and disinfectant-generating reactants (of the disinfectant catalyst) combine and generate a disinfectant (such as chlorine dioxide). The disinfectant (e.g., chlorine dioxide) in vapor phase is generated and diffuses through the outer layer of the module 200 (e.g., the permeable membrane-such as a polypropylene mesh outer layer) and is delivered to the air flow within the system (e.g., HVAC system, electrostatic precipitator in system, etc.). A constant supply of liquid to the modules 200 provides continuous, on-demand disinfectant vapor generation within the system.

Further, vibration of modules may occur due to the obstructed flow of air around the modules. By this process, high turbulence and eddy formation also occurs, which will allow mixing of disinfectant (e.g., chlorine dioxide) in air, thereby increasing the probability of neutralization of air (and surface) borne pathogens. Pathogens may also be neutralized with impaction on surfaces of the modules (or impaction on other surfaces that have been exposed to the generated disinfectant).

Further, as the disinfectant catalyst is consumed, the size, shape, and/or volume, etc. of the disinfectant catalyst 202 will change (reduce). In certain embodiments, as this occurs, the outer layer 204 may include a material that will constrict to the disinfectant catalyst as it reduces. As one non-limiting example, the outer layer may be in the form of a sleeve including a polypropylene mesh. As disinfectant catalyst is consumed, the polypropylene mesh sleeve conforms to the core size of the disinfectant catalyst rod of FIG. 1C as it reduces, thereby promoting constant contact and reactivity with the supplied liquid, thereby providing continuous disinfectant generation. The solid disinfectant rod configuration offers core geometry to ensure sufficient contact between the wetted mesh sleeve and the disinfectant catalyst material.

Additionally, the present system provides simple maintenance and replacement of modules through connection mounts that can be associated with the structure described above. As disinfectant-generating reactants of the disinfectant catalyst are consumed and depleted (or ultimately exhausted), individual modules can be easily replaced by removing the outer layer with spent disinfectant catalyst core and replacing with a new module of disinfectant catalyst/outer layer. In certain embodiments, the inlet/outlet connections require minimal tooling and can slide into place through slots in the frame structure, through a snap on mechanism, or other simple connection structure. In an alternate embodiment, rather than removing individual modules as or after the disinfectant catalyst is consumed, one may remove and replace entire panel structures including multiple modules.

Another aspect of the present invention is directed to the geometries of the modules. In a general aspect, the modules (or the components thereof such as the disinfectant catalyst and any outer layer) may be of a shape that allows for increased surface area of each module, and allows for vibration of the module. Due to vibrations (which may be caused by the passage of airflow around each module), the area of the plume of disinfectant vapor phase that is shed from the module may be increased, thus leading to more efficient neutralization of pathogens. Increased surface area may also contribute to an increased amount of disinfectant that is shed into the airflow- and provides additional surface that can be impacted by pathogens in the airflow. Additionally, the geometries of the modules can also contribute to increased efficiency (e.g., irregular shapes can create more turbulence around the modules during vibration). This results in a greater dispersion of disinfectant in the vapor phase. The various geometries that may be used to increase efficiency may be geometries of the disinfectant catalyst, or of the outer layer, or both.

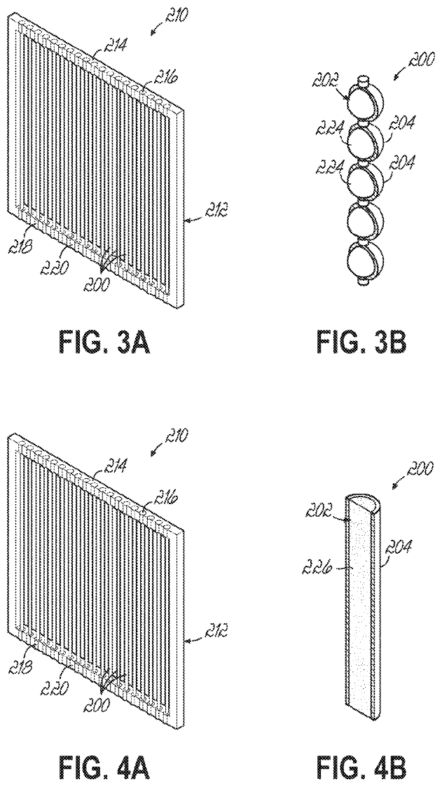

For example, the geometry of the disinfectant catalyst rod of the particular embodiment shown in FIG. 1C, may reduce the desired vibrational amplitude of the collection surface. To alleviate this, an alternate solid disinfectant catalyst core configuration is presented in FIGS. 2A and 2B. This configuration consists of a plurality of disinfectant beads 222 enclosed in an outer layer 204—such as a polypropylene mesh sleeve. In this embodiment, the disinfectant beads provide additional surface area for contact with the wetted mesh allowing for increased disinfectant generation. Additionally, the loose beads reduce structural stiffness (as compared to the rod structure of FIG. 1C) and thus allow for induced vibrations of the collection surfaces. And, the disinfectant bead core provides increased surface area for disinfectant generation as well as pathogen capture. Like the configuration shown in FIG. 1C, the outer polypropylene mesh sleeve will continue to provide continuous liquid-catalyst contact due to conforming to the catalyst material as the catalyst is consumed. Additional embodiments may be used where solid disinfectant cores are utilized in conjunction with the catalyst beads in an alternating orientation.

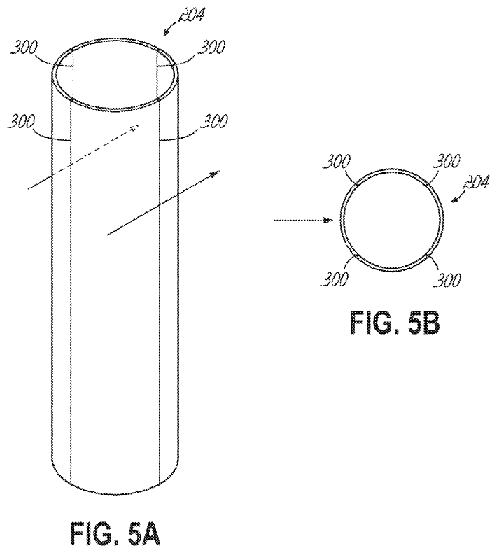

Yet another alternate solid disinfectant catalyst core configuration is presented in FIG. 3B (which may be used in the panel apparatus of FIG. 3A). This configuration consists of a plurality of a single row of disinfectant beads 224 enclosed in an outer layer 204—such as a polypropylene mesh sleeve. In this embodiment, the disinfectant beads provide additional surface area for contact with the wetted mesh allowing for increased disinfectant generation.

Additionally, the beads do not add additional structural stiffness and allow for induced vibrations of the collection surfaces. And the disinfectant bead core provides increased surface area for disinfectant generation as well as particulate capture. Even as compared to FIGS. 2A and 2B, this bead design may further increase gas generation as well gas-air mixing due to higher surface area and additional turbulence around the region in between any two beads. Like the configurations shown in FIGS. 1C and 2A/2B, the outer polypropylene mesh sleeve will continue to provide continuous liquid-catalyst contact due through conforming to the core material as it is consumed.

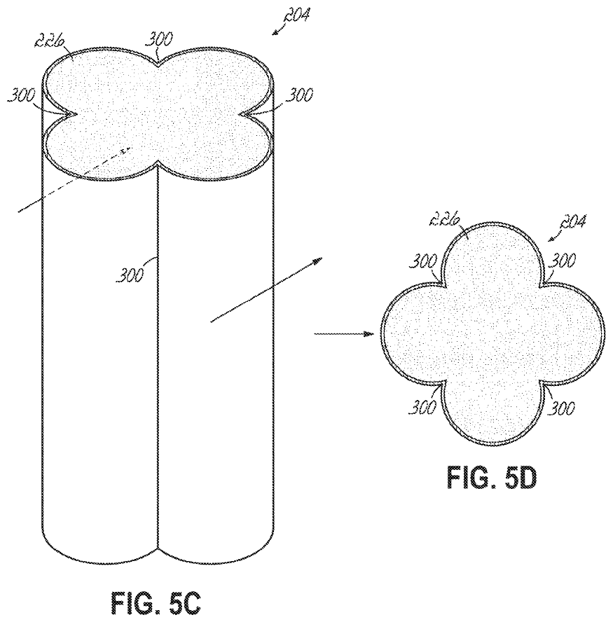

Yet another alternate solid disinfectant catalyst core configuration is presented in FIG. 4B (which may be used in the panel apparatus of FIG. 4A). This configuration consists of a packed powdered disinfectant catalyst 226 enclosed in an outer layer 204—such as a polypropylene sleeve. Like the beads described above, the powder form does not add additional structural stiffness and it allows for induced vibrations of the collection surfaces. Like the configurations shown in FIGS. 1C and 2A/2B, the outer polypropylene mesh sleeve will continue to provide continuous liquid-catalyst contact due through conforming to the core material as it is consumed.

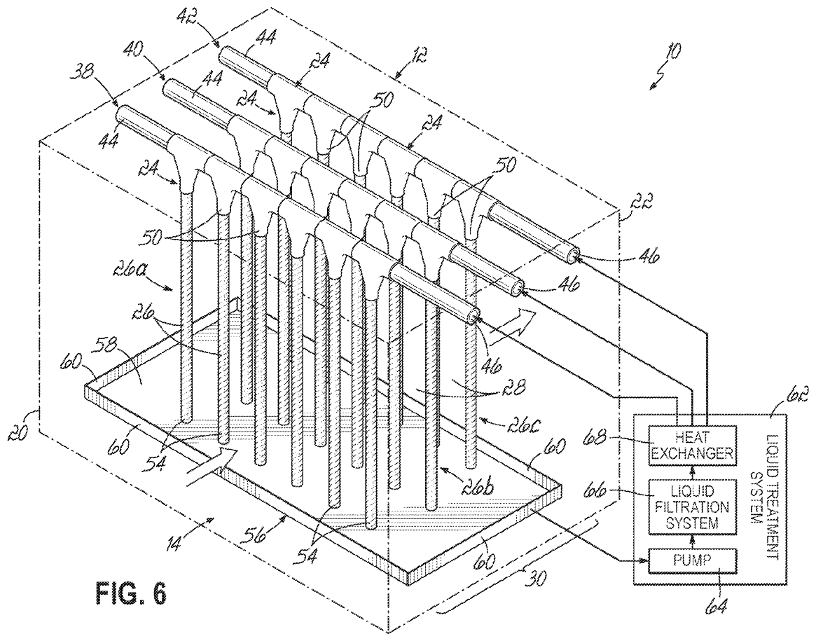

It may be recognized by those of ordinary skill in the art that the powder embodiment described with respect to FIG. 4B will conform to the shape of the outer layer. And so, if the outer layer is a substantially cylindrical sleeve, various geometries that can be useful in generating the additional turbulence and eddies described above may not be obtained. However, in certain embodiments, this may be rectified by having the outer layer provide the desired geometries. For example, a propylene mesh sleeve (which would expand when packed with disinfectant catalyst powder, and which would constrict to remain in contact with powder as it was depleted) could be seamed in order to provide portions that do not expand, and portions that do expand. A non-limiting example of this is shown in FIGS. 5A and 5B, which show a sleeve, and a cross section thereof, in non-filled form. As can be seen, the sleeve 204 includes a plurality of seamed portions running longitudinally along the body of the sleeve. These seamed areas will not expand, whereas the portions in between the seams 300 can expand. This can be seen in FIGS. 5C and 5D, which shows the same sleeve (and cross-section thereof) now filled with powdered disinfectant catalyst. As can be seen, the expandable portions of the sleeve have expanded to provide an overall irregular shape to the outer layer. This can then provide additional surface layer, and cause enhanced turbulence and eddies for greater dispersion of disinfectant in vapor phase (as described above).

Yet another approach to delivering the disinfectant can be developed by using the outer layer itself (e.g., mesh sleeve) as a wick that disperses the disinfectant by using capillary action. In both the vibrating precipitator and crossflow precipitator systems, a braided rope has been shown to be an excellent transport media for liquids since the capillary action keeps the sleeve wet which produces uniform dispersion from the sleeve. In addition, the capillary action also produces a wicking effect that could be used to disperse the disinfectant. It should be noted that that disinfectants can be selected that will vaporize in the air stream. The net result is dispersion of the disinfectant without having to pump the liquid into the mesh sleeves.

Further, additional reaction between liquid and disinfectant-generating reactants may be initiated by placing a water reservoir in the bottom of the system, or apparatus (such as the bottom of the panel structure. The wicking action of the outer layer (e.g., hydrophilic membrane) will allow water to flow up from the reservoir promoting reaction and generation of disinfectant gas from lower part of the module. This mechanism thus provides a steady and stable supply of disinfectant vapors.

Various materials may be incorporated into the outer layer 204 of the modules 200. In certain embodiments, the outer layer may include a hydrophilic material. And, use of a hydrophilic material may promote the interaction of the liquid with the disinfectant catalyst. However, in other embodiments, the outer layer could include a hydrophobic material. By using a hydrophobic material, it will take longer for liquid (water) to pass along the material (as compared to a hydrophilic material), and thus longer for it to come into contact with the disinfectant catalyst. This can extend the life of the disinfectant catalyst (i.e., the catalyst won't generate disinfectant as rapidly because it is not contacted by liquid as easily, and thus won't be exhausted as rapidly). In yet another embodiment, the outer layer may include both hydrophilic and hydrophobic materials (e.g., a weave of such materials). By manipulating the amount of hydrophilic material versus hydrophobic material in the outer layer, one could control the rate at which the disinfectant catalyst is used, and thus the lifespan of each module (e.g., higher percentages of hydrophobic materials equals a longer lifespan, whereas higher percentages of hydrophilic materials equals a shorter lifespan).

Apart from the various embodiments of the outer layer described above, one could also control rate of disinfectant generation and disinfectant dispersal by using a completely non-permeable sleeve around the disinfectant catalyst, and then providing holes in the sleeve to allow introduction of liquid and dispersal of disinfectant. Rate could be controlled by size and/or number of holes.

As described above with respect to FIGS. 1A-1C, a module 200 (or modules) including a disinfectant-generating reactant 202 (which may be referred to herein as a disinfectant catalyst 202 or disinfectant catalysts) are at least partially disposed within an outer layer 204 (such as a permeable membrane sleeve). In particular, the illustrated embodiment of FIG. 1C includes a solid disinfectant catalyst core encased in an outer layer (e.g., an elastic polypropylene mesh outer layer). And, as mentioned above, module(s) or plurality of modules arranged within a housing may be introduced into any type of system to treat air within the particular system. For example, such systems where the modules may be used include (but are not limited to) HVAC systems or any air filtration type system (such as electrostatic precipitators). By positioning the modules within the airflow path of such systems, one may cause capture and/or neutralization of pathogens in the airflow. Thus, as a nonlimiting example, the combination of solid disinfectant catalyst 202 and outer layer 204 (a disinfectant catalyst/outer layer module 200) can be used in a precipitator device (such as the precipitator device 10 shown in FIGS. 6 and 7)

In that regard, in a vibrating wet precipitator 10 (VWP) of the type shown in FIGS. 6 and 7, particulate-laden hot gas flows through an array 30 of vertical wet cords 26 stretched within duct 12. This gas may also include any number and/or type of pathogens. FIGS. 6 and 7 shows an exemplary embodiment of precipitator device 10 with the plurality of cords 26 fixed at top and bottom with a desired tension. There are three rows of cords labeled 26(a), 26(b) and 26(c) which are part of sieves 38, 40 and 42, respectively. The plurality of sieves 24 are arranged to define the plurality of gaps 28 through which the exhaust flows from a duct inlet 20 to a duct outlet 22.

The sieve assembly 16 as shown in FIG. 6 includes first, second, and third sieve arrangements 38, 40, 42, respectively. Each of the sieve arrangements 38, 40, 42, includes sieves 24 offset and parallel from each other along a linear row. The plurality of sieves 24 are oriented generally vertically/longitudinally and, as such, perpendicular to the transverse flow direction of the exhaust. While the sieves 24 are distributed about the flow chamber 14 generally evenly to define like gaps 28, it will be appreciated that more or less sieves 24 may be used with varying orientation and placement within the duct 12.

Each sieve arrangement 38, 40, 42 includes a generally horizontally extending support member 44, which defines a liquid supply conduit 46 extending therethrough. The support member 44 supports the generally vertical orientation of the elongated cords 26 and is configured to introduce the liquid therein to define a sieve inlet 50.

The vibrating wet precipitator device 10 further includes a liquid collector 56 positioned proximate to the sieve outlets 54 for collecting the liquid being discharged from the sieve outlets 54. In addition, the elongated cords 26 connect to the liquid collector 56 as another frame member 56. As such, the elongated cords 26 extend between the frame members 44, 56.

The liquid collector 56 is in the form of a tray 56 that includes a bottom 58 and surrounding sidewalls 60 configured to guide the liquid to a liquid treatment system 62. The liquid treatment system 62 includes a pump 64, a filtration system 66, and a heat exchanger 68. The pump 64 is configured to direct the liquid from the liquid collector 56 to the filtration system 66, which is configured to remove particulate emissions from the liquid. The pump 64 then continues to direct the liquid through the heat exchanger 68 for recovering thermal energy from the liquid. While the liquid may be removed from the precipitator device 10, the liquid may also be redirected back into the liquid supply conduit 46 for reuse through the elongated cords 26.

FIG. 7 shows an exemplary embodiment of a vibrating wet precipitator having three arrays 30, 32 and 34 of cords 26. The number of arrays, as well as the number of cords per array, can be modified to maximize collection efficiency.

The elongated cords 26 of the precipitator of FIGS. 6 and 7 are used primarily to remove particulate contaminants. In order to remove pathogen contaminants, modules 200 in accordance with the principles described herein can be added to the precipitator along with, or instead of, the cords 26 described with respect to FIGS. 6 and 7.

In other words, a precipitator may include a plurality of disinfectant catalyst/outer layer modules 200—each one fixed at a top end 206 and a bottom end 208 to a surface of the precipitator, or to a component of the precipitator. Similar to that described with respect to FIGS. 6 and 7, the plurality of disinfectant catalyst/outer layer modules 200 may be arranged to define a plurality of gaps through which air flows from a duct inlet to a duct outlet. For example, the assembly may include first, second, and third module arrangements where each of the module arrangements includes modules (disinfectant catalyst/outer layer combinations) offset and parallel from each other along a linear row. The plurality of modules may be oriented generally vertically/longitudinally and, as such, perpendicular to the transverse flow direction of the air. While the modules may be distributed about the flow chamber generally evenly to define like gaps, it will be appreciated that more or less modules may be used with varying orientation and placement within the duct.

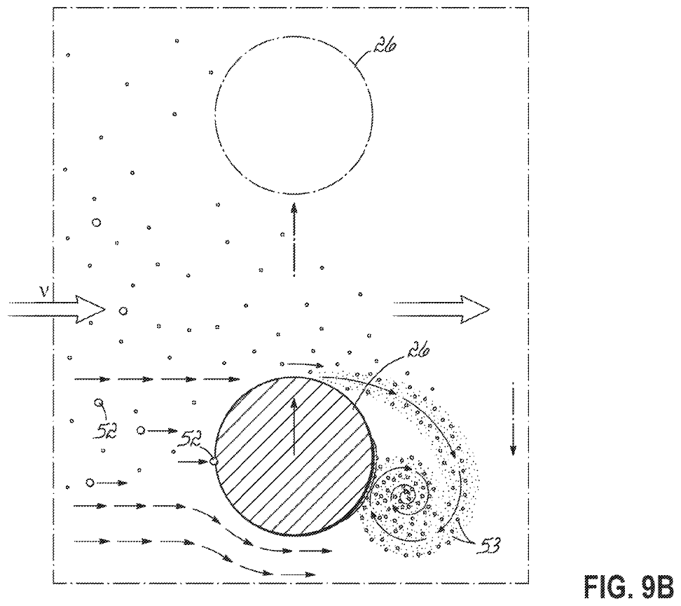

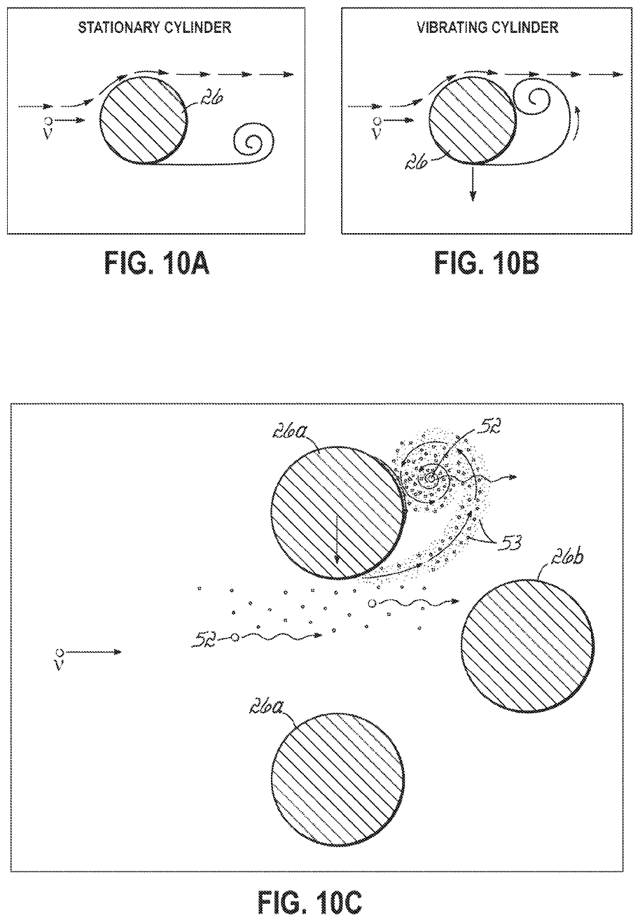

Next, as described above, various embodiments of the modules may vibrate due to the passage of air, which enhances the ability of the disinfectant to capture and or neutralize pathogens in the air stream. In use, as shown in FIGS. 9A-10C, (and using the embodiment including elongated cords as an example), the flow of the air forces the elongated module 26(a) to vibrate between first and second positions. Some pathogens 52 impact the modules 26(a), which neutralize the pathogens thereon. However, other pathogens 53 tend to follow the flow of the air around the vibrating modules 26(a). To this end, the modules 26(a) generate the trailing vortices and effectively capture and neutralize these pathogens within each trailing vortex. The vortices from modules 26(a) will impact modules 26(b) which, in turn impact modules 26(c), all of which will be vibrating as shown by FIGS. 9A and 9B. The beneficial flow turbulence is further enhanced by smaller-amplitude vibrations of the modules ropes in the flow direction; these vibrations have higher frequencies than those in the lateral direction. The liquid flowing along the modules 26 will contact the disinfectant catalyst, and it will disperse to a greater extent than it otherwise would due to the vibrations.

While the present invention has been illustrated by the description of one or more embodiments thereof, and while the embodiments have been described in considerable detail, they are not intended to restrict or in any way limit the scope of the appended claims to such detail. The various features shown and described herein may be used alone or in any combination.

What is claimed is:

1. A device for removal of pathogens including a disinfectant module, wherein the disinfectant module includes a disinfectant catalyst, wherein at least a portion of the disinfectant catalyst may be used in a vapor phase.

2. The device of claim 1, wherein the module includes a disinfectant catalyst disposed at least partially within an outer layer.

3. The device of claim 2, wherein the disinfectant catalyst is in a form chosen from a powder, a rod, and a plurality of beads.

4. The device of claim 2, wherein the outer layer includes a hydrophilic material, a hydrophobic material, or a mixture of hydrophilic and hydrophobic materials.

5. The device of claim 2, wherein at least one or more portions of the outer layer are expandable and contractable.

6. A device for removal of pathogens including a plurality of disinfectant modules as described herein.

7. The device of claim 6, wherein each module of the plurality of modules includes a disinfectant catalyst disposed at least partially within an outer layer.

8. The device of claim 7, further including a housing, wherein each module of the plurality of modules is disposed therewithin.

9. The device of claim 8, wherein each module of the plurality of modules has a top end and a bottom end, and wherein the device further includes a fluid conduit in communication with the plurality of modules.

10. The device of claim 9, wherein the fluid conduit is in direct communication with the top end of the plurality of modules.

11. The device of claim 9, wherein the fluid conduit is in direct communication with the bottom end of the plurality of modules.
